(19) United States
(12) Patent Application Publication
(10) Pub. No.: US 2006/0094785 A1
(43) Pub. Date: May 4, 2006

(54) Use of amino acids for treatment of various conditions

(76) Inventor: Thomas J. Guttuso Jr, Snyder, NY (US)

Correspondence Address: Edwin V Merkel;Nixon Peabody, Clinton Square, PO Box 31051, Rochester, NY, 14603, US

(21) Appl. No.: 10/519,598

(22) PCT Filed: Jul. 14, 2003
(86) PCT No.: PCT/US03/21785

§ 371 Date: Sep. 23, 2005

Related U.S. Application Data
(60) Provisional application No. 60/395,975, filed on Jul. 12, 2002.

(51) Int. Cl. A61K 31/198

(52) U.S. Cl. 514/561; 514/562; 514/567

(57) ABSTRACT

A method of treating a patient for a condition characterized by symptoms that can be alleviated by interfering with or supplementing the activity of endogenous ligands on the a2S subunit of a voltage gated calcium channel, said method comprising: administering to a patient experiencing the condition an amount of one or more of L-norleucine, L-isoleucine, L-alloisoleucine, L-methionine, Lleucine, 2-cyclohexylglycine, 2-phenylglycine, 2-amino-2-norbornane carboxylic acid, 1-aminocyclohexane carboxylic acid, 2-aminoheptanoic acid, 2-aminocaprylic acid, and 2-aminononanoic acid under conditions effective to treat the condition, wherein when the condition is a hot flash or a symptom of hormonal variation, the compound is not L-leucine.

This application claims the priority benefit of U.S. Provisional Patent Application Ser. No. 60/395,975 filed Jul. 12, 2002, which is hereby incorporated by reference in its entirety.

### FIELD OF THE INVENTION

The present invention relates generally to the use of a therapeutically effective amount of various compounds, or compositions containing such compounds, to treat conditions that are believed to be mediated by the α₂δ subunit of voltage gated calcium channels (“VGCC”).

### BACKGROUND OF THE INVENTION

Gabapentin and various γ-amino-butyric acid (GABA) derivatives or analogs have been reported to be useful for treating a number of conditions. These include: hot flashes and symptoms of hormonal variation (U.S. Pat. No. 6,310,098 to Guttuso, Jr.); seizures (U.S. Pat. No. 6,359,169 to Silverman et al.); vertigo and migraine headaches (U.S. Pat. No. 6,333,352 to Derakhshan); chronic pain disorders (U.S. Pat. No. 6,316,638 to Bryans et al.); symptoms of neurodegenerative diseases such as Parkinson's Disease, Alzheimer's Disease, Huntington's Disease, Multiple Sclerosis, Amyotrophic Lateral Sclerosis, etc. (U.S. Pat. No. 6,359,169 to Silverman et al.); restless legs syndrome (U.S. Pat. No. 6,316,638 to Bryans et al.); tremor disorders (Magnus, “Nonepileptic Uses of Gabapentin,” Epilepsia, 40:S66-S72 (1999)); nausea (U.S. Pat. No. 5,916,903 to Viner); anxiety and depression disorders and insomnia (U.S. Pat. No. 6,372,792 to Chouinard and U.S. Pat. No. 6,306,910 to Magnus et al.); various sleep disorders (U.S. Pat. No. 6,586,478 to Ackman et al.); both irritable bowel syndrome and inflammatory bowel syndrome (U.S. Pat. No. 6,242,488 to Bueno et al.) as well as gastrointestinal damage caused by drugs and alcohol (U.S. Pat. No. 6,426,368 to Bueno et al.); obsessive compulsive disorders, generalized anxiety disorders, and impulse control disorders, which may include gambling disorders, compulsive eating, body dysmorphic disorders, hypochondriasis, pathological grooming conditions, kleptomania, pyromania, and attention defecit hyperactivity disorder (U.S. Pat. No. 6,462,084 to Dewey et al.); and drug addiction (U.S. Pat. No. 6,541,520 to Dewey et al.).

Gabapentin and GABA derivatives or analogs have been shown to bind to a single site in the brain with high affinity, the α₂δ subunit of VGCC (Bryans et al., “3-Substituted GABA Analogs with Central Nervous System Activity: A Review,” Med. Res. Rev. 19:149-177 (1999)). It is believed that their interaction with this site is responsible for their clinical efficacy for multiple indications such as those listed above.

Despite the success of gabapentin and GABA derivatives or analogs in treating numerous conditions, gabapentin use is associated with various side effects, most often sleepiness and dizziness, leading to an approximately 13 percent patient withdrawal rate (Backonja et al., “Gabapentin for the Symptomatic Treatment of Painful Neuropathy in Patients with Diabetes Mellitus: A Randomized Controlled Trial,” JAMA 280:1831-1836 (1998); Rowbotham et al., “Gabapentin for the Treatment of Postherpetic Neuralgia: A Randomized Controlled Trial,” JAMA 280:1837-1842 (1998)). Finally, as a prescription drug, use of gabapentin is often too costly and poorly accessible for some patients who are in need of an effective treatment for their condition(s). It would be desirable, therefore, to identify other compounds that are well tolerated and substantially free of side effects, less expensive than existing therapies, and readily accessible.

The present invention overcomes these and other deficiencies in the art.

### SUMMARY OF THE INVENTION

The present invention relates to a method of treating a patient for a condition characterized by symptoms that can be alleviated by interfering with or supplementing the activity of endogenous ligands on the α₂δ subunit of a voltage gated calcium channel (“VGCC”), the method including the step of administering to a patient experiencing the condition an amount of one or more of L-norleucine, L-isoleucine, L-alloisoleucine, L-methionine, L-leucine, 2-cyclohexylglycine, 2-phenylglycine, 2-amino-2-norbornane carboxylic acid, 1-aminocyclohexane carboxylic acid, 2-aminoheptanoic acid, 2-aminocaprylic acid, and 2-aminononanoic acid under conditions effective to treat the condition, wherein when the condition is a hot flash or a symptom of hormonal variation, the compound is not L-leucine.

Another aspect of the present invention relates to a composition in a single unit dosage form that includes: a pharmaceutically or organoleptically acceptable carrier, and one or more compounds selected from the group consisting of 2-cyclohexylglycine, 2-phenylglycine, 2-amino-2-norbornane carboxylic acid, 1-aminocyclohexane carboxylic acid, 2-aminoheptanoic acid, 2-aminocaprylic acid, 2-aminononanoic acid, L-norleucine, L-isoleucine, L-alloisoleucine, L-methionine, and L-leucine, wherein the single unit dosage form includes an amount of the one or more compounds which is effective to treat a condition characterized by symptoms that can be alleviated by interfering with the activity of endogenous ligands on the α₂δ subunit of a voltage gated calcium channel.

The present invention affords effective treatment of a number of conditions or disorders, whereby any number of the above-identified compounds can be administered individually or in combination, either alone or in the form of a pharmaceutical composition or a nutrition supplement, for purposes of treating the various conditions or disorders. The compounds disclosed herein are believed to act on the α₂δ subunit of the VGCC, the site where gabapentin and GABA analogs and derivatives are believed to have their effect. However, unlike gabapentin and GABA analogs and derivatives, the compounds disclosed herein for use in accordance with the present invention are believed to be well tolerated and (unless otherwise noted) substantially free of side effects, less expensive, and readily accessible.

### DETAILED DESCRIPTION OF THE INVENTION

The present invention relates to the treatment of various conditions using one or more of the following compounds: 2-cyclohexylglycine or H-cyclohexyl-Gly-OH (Bachem Bioscience, Inc., King of Prussia, Pa.), 2-phenylglycine (Aldrich Chemical Company, Inc., Milwaukee, Wis.), 2-amino-2-norbornane carboxylic acid (Sigma Chemical Co., St. Louis, Mo.), 1-aminocyclohexane carboxylic acid (Sigma Chemical Co.), 2-aminoheptanoic acid (Sigma Chemical Co.), 2-aminocaprylic acid (Sigma Chemical Co.), 2-aminononanoic acid (Sigma Chemical Co.), L-norleucine (Peptides International, Inc., Louisville, Ky.), L-isoleucine (Peptides International, Inc.), L-alloisoleucine (Sigma Chemical Co.), L-methionine (Peptides International, Inc.), and L-leucine (Peptides International, Inc.).

Each of the above-identified compounds is commercially available in substantially pure form (e.g., 95% or higher) or, depending on the compound, as a racemic mixture. Both the substantially pure compounds and the compounds present as a racemic mixture are useful in accordance with the present invention.

The compounds can be administered alone or as a component of a composition in the form of a pharmaceutical or nutritional supplement

Of the above-listed compounds, L-norleucine, L-isoleucine, L-methionine, and L-leucine are naturally occurring amino acids and, therefore, can be administered in the form of a nutritional supplement.

The remaining compounds, L-alloisoleucine, 2-cyclohexylglycine, 2-phenylglycine, 2-amino-2-norbornane carboxylic acid, 1-aminocyclohexane carboxylic acid, 2-aminoheptanoic acid, 2-aminocaprylic acid, and 2-aminononanoic acid are non-naturally occurring compounds and, therefore, can be administered in the form of a pharmaceutical.

Effective amounts of the compound(s) will depend upon the mode of administration, frequency of administration, and the type of pharmaceutical or nutritional supplement composition used to deliver the compound into a patient. Generally, effective amounts of such compounds will be about 0.01 to about 300 mg/kg·body wt. per day, preferably about 0.1 to about 200 mg/kg·body wt. per day, more preferably about 1 to about 100 mg/kg·body wt. per day. Typical daily doses will be from about 10 to about 5000 mg per day for an average adult patient of normal weight. While individual needs vary, determination of optimal ranges of effective amounts of each compound is within the abilities of those of skill of the art.

The nutritional and/or pharmaceutical composition will include one or more of the above-identified compounds in combination with a suitable carrier. In the case of the pharmaceutical composition, the carrier is a pharmaceutically acceptable carrier. In the case of a nutritional supplement, the carrier is an organoleptically suitable carrier.

For compositions of the present invention, it is preferable that such compositions are in the form of a single unit dosage form that contains an amount of the one or more compounds effective to treat the condition to be alleviated.

Other compositions encompassed by the present invention include those containing two or more of the above-identified compounds in combination with suitable carriers. The compositions of the present invention may exclude other active ingredients or, alternatively, the compositions can be administered in combination with other therapeutic regimen that are known in the art, whether now known or hereafter developed.

The nutritional supplement and/or pharmaceutical composition can also include suitable excipients, or stabilizers, and can be in solid or liquid form such as, tablets, capsules, powders, solutions, suspensions, or emulsions. Typically, the composition will contain from about 0.01 to 99 percent, preferably from about 5 to 95 percent of active compound(s), together with the carrier.

The one or more compound(s), when combined with a suitable carrier and any excipients or stabilizers, whether administered alone or in the form of a composition, can be administered orally, parenterally, subcutaneously, transdermally, intravenously, intramuscularly, intraperitoneally, by intranasal instillation, by implantation, by intracavitary or intravesical instillation, intraocularly, intraarterially, intralesionally, or by application to mucous membranes, such as, that of the nose, throat, and bronchial tubes (i.e., inhalation).

For most therapeutic purposes, the one or more compound(s) can be administered orally as a solid or as a solution or suspension in liquid form, via injection as a solution or suspension in liquid form, or via inhalation of a nebulized solution or suspension.

The solid unit dosage forms can be of a conventional type. The solid form can be a capsule, such as an ordinary gelatin type containing the one or more compound(s) and a carrier, for example, lubricants and inert fillers such as, lactose, sucrose, or cornstarch. In another embodiment, these compounds are tableted with conventional tablet bases such as lactose, sucrose, or cornstarch in combination with binders like acacia or gelatin, disintegrating agents such as cornstarch, potato starch, or alginic acid, and a lubricant such as stearic acid or magnesium stearate.

For injectable dosages, solutions or suspensions of the one or more compound(s) can be prepared in a physiologically and pharmaceutically acceptable diluent as the carrier. Such carriers include sterile liquids, such as water and oils, with or without the addition of a surfactant and other pharmaceutically and physiologically acceptable components, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the compound in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

With respect to the naturally occurring amino acids described above, a patient may alternatively increase administration of these compounds by modifying his or her diet accordingly. Thus, by consuming foods that are high in these compounds, a patient may increase his or her daily intake of these amino acids to produce a therapeutic effect with respect to the various indications listed above. Foods high in L-norleucine include vegetables, especially green leafy vegetables; foods high in L-leucine include eggs, fish, lentils, poultry, beef, seeds, soy, wheat, almonds, dairy, beans, and brown rice; and foods high in L-methionine include fish, eggs, dairy, beans, beef, garlic, onion, lentils, and soybeans. Because L-leucine is naturally converted by the body into L-isoleucine and L-norleucine, the above foods rich in L-leucine can increase the in vivo concentration of L-isoleucine and L-norleucine.

Even though a patient may increase intake of these naturally occurring amino acids by diet, nutritional supplements and/or pharmaceutical compositions can also be administered in accordance with the present invention.

The various conditions that can be treated in accordance with the present invention are those conditions characterized by symptoms that can be alleviated by interfering with or supplementing the activity of endogenous ligands on VGCC, particularly the α₂δ subunit of the VGCC. Without being bound by belief, regardless of the route and form of administration, it is believed that the above-listed compounds can be used to treat a variety of conditions by virtue of their binding activity on the α₂δ subunit of the VGCC.

The various conditions that can be treated in accordance with the present invention include, without limitation, hot flashes and symptoms of hormonal variation; seizures; vertigo and migraine headaches; chronic pain disorders; symptoms of neurodegenerative diseases including, without limitation, the symptoms of Parkinson's Disease, Alzheimer's Disease, Huntington's Disease, Multiple Sclerosis, and Amyotrophic Lateral Sclerosis; tic disorders; tremor disorders; nausea; cough; hiccups; asthma; hyperhidrosis; sleep disorders; fatigue; fibromyalgia; premature labor; preeclampsia and eclampsia; irritable bowel syndrome and inflammatory bowel disease; gastrointestinal damage caused by drugs and alcohol; drug addiction; obsessive compulsive disorders, generalized anxiety disorders, and impulse control disorders; and attention defecit hyperactivity disorder.

Thus, one aspect of the present invention relates to a method of treating the above-listed conditions in a patient which is carried out by administering an amount of the one or more of the above-identified compounds to a patient experiencing symptoms of one or more of the above-listed conditions in a manner effective to treat those symptoms. Alternatively, an agent that is converted by the body into one of the above-identified compounds can be administered to the patient. By treating a particular condition, the present invention encompasses either reducing the number of symptomatic events, reducing the severity of symptomatic events, or both.

The patient to be treated is any mammalian patient, preferably a human patient, either female or male.

With respect to treatment of hot flashes and symptoms of hormonal variation, it should be appreciated to those of skill in the art that the ultimate cause of hot flashes can, of course, be markedly different for male and female patients. For example, in female patients the hot flash is a primary symptom resulting from menopausal or postmenopausal hormonal variation. However, the hot flash can also be drug-induced by anti-estrogen compounds (e.g., tamoxifen, raloxifene, leuprolide acetate, etc.) or surgically-induced by removal of estrogen-producing tissues (e.g., total abdominal hysterectomy, bilateral salpingo-oophorectomy, etc.). In male patients, the hot flashes typically occur as a side-effect of androgen-deprivation therapy for metastatic prostate cancer. They can be either surgically-induced (e.g., bilateral orchiectomy) or drug-induced (e.g., treatment with a gonadotrophin-releasing-hormone agonist, leuprolide acetate, etc.). The present invention is directed to treatment of hot flashes and associated symptoms of hormonal variation that are affiliated with these and other causes thereof.

Nausea and emesis (or vomiting) are often induced by stimulation of either the chemoreceptor trigger zone or the emesis center in the central nervous system. Such stimulation can be caused by afferent stimulation (e.g., tactile pharyngeal impulses, labrynthine disturbances, motion, increased intracranial pressure, pain, distention of viscera, or psychologic factors) or blood born emetic substances (e.g., as seen during pregnancy or during episodes of premenstrual syndrome, cancer chemotherapy, uremia, radiation therapy, electrolyte and endocrine disturbances, or the presence of chemical emetic substances). Nausea and vomiting are also common post-operative side effects resulting from the use of anesthetics. The present invention is directed to treatment of nausea and emesis that are affiliated with these and other causes thereof.

Fatigue includes that associated with chemotherapy administration, with other medication therapy or toxin exposure, with a disease state, and that occurring without known cause.

Chronic pain disorders can include both neuropathic and non-neuropathic pain disorders. Examples include, but are not limited to, diabetic neuropathy, post-herpetic neuralgia, trigeminal neuralgia, occipital neuralgia, carpal tunnel syndrome, chronic headache conditions, chronic backache conditions, arthritis, bursitis, tendonitis, muscle cramping, myositis, and myopathy conditions.

Sleep disorders can include, generally, both dyssomnias and parasomnias. Exemplary sleep disorders to be treated include, without limitation, insomnia, sleep apnea, REM sleep disorders, restless legs syndrome, periodic leg movements of sleep, and night terrors.

Tremor disorders include but are not limited to those associated Parkinson's Disease, Essential Tremor, Intention Tremor, Rubral Tremor, Orthostatic Tremor, Physiologic Tremor, Cerebellar Tremor, drug-induced tremor, idiopathic tremor, cerebral ischemia, tardive dyskenesia, spasticity, and other disorders associated with dopaminergic neuron malfunction. It has been demonstrated the GABA-ergic neurons project onto dopaminergic neurons of the ventral tegmental area and are inhibitory in nature. Therefore, it is evident that modifying the activity of GABAergic neurons may affect the activity of dopaminergic neurons and, hence, be useful to treat conditions in which the dopaminergic neurons are implicated.

Tic disorders include can include common simple motor tics such as eye blinking, neck jerking, shoulder shrugging, facial grimacing, and coughing, common simple vocal tics such as throat clearing, grunting, sniffing, snorting, barking, common complex motor tics such as facial gestures, grooming behaviors, jumping, touching, stamping, and smelling of objects; common complex vocal tics such as repeating words or phrases out of context, coprolalia (use of socially unacceptable words, frequently obscene), palilalia (repeating one's own sounds or words), and echolalia (repeating the last heard sound, word, or phrase); and multiple tic disorders such as Tourette's syndrome.

Symptoms of neurodegenerative diseases or disorders that can be treated include bradykinesia, rigidity, tremors, postural instability, depression, and other symptoms associated with Parkinson's Disease; dementia and other symptoms associated with Alzheimer's Disease; chorea dystonia, dementia, athetosis, and other symptoms associated with Huntington's Disease; spasticity, weakness, optic neuritis, and other symptoms associated with Multiple Sclerosis; and muscle atrophy, fasciculation of muscles, spasticity, weakness, optic neuritis, and other symptoms associated with Amyotrophic Lateral Sclerosis.

Migraine is a disorder characterized by persistent headache, which may be severe, which may be associated with visual and gastrointestinal disturbances, and which may also be recurrent. The head pain associated with migraine may be unilateral or generalized. Migraine can recur at a frequency that varies widely, from daily events to once in several months. An untreated acute migraine episode can endure for as long as many hours or several days.

Cough can be a result of infection, drug-induced, secondary to asthma or emphysema, or idiopathic.

Hiccups can be drug-induced, surgically-induced, or idiopathic.

Hyperhidrosis can be drug-induced, surgically-induced (also known as compensatory sweating), secondary to hormonal fluctuations, or idiopathic.

Irritable Bowel Syndrome is a functional bowel disorder in which abdominal pain is associated with defecation or a change in bowel habits. IBS has elements of an intestinal mobility disorder, a visceral sensation disorder, and a central nervous system disorder. While the symptoms of IBS have a physiological basis, no clear mechanism unique to IBS has been identified. Rather, the same mechanisms that cause occasional abdominal discomfort in healthy individuals seems to operate to produce the symptoms of IBS. Persons with IBS exhibit hypersensitivity, particularly hyperalgesia, in response to painful distensions in the small bowel and colon and to normal intestinal function. There are also increased or unusual areas of visceral pain, often worsened by meals and alleviated upon defecation. Together, the gastrointestinal disorders of IBS and inflammatory bowel disease encompass a wide range of disease states, including without limitation Crohn's disease, ileitis, ischemic bowel disease, ulcerative colitis, dyspepsia, gastroesophogeal reflux for functional bowel disorders, and other forms of visceral pain.

Gastrointestinal damage caused by drugs and alcohol can be take the form of mild dyspepsia, gastritis, peptic ulcer disease, as well as more severe gastrointestinal complications such as bleeding and perforation. It is well known that symptoms from drug and alcohol withdrawal can include, among others, tremors, anxiety, convulsions, hallucinations, and confusion.

Obsessive compulsive disorders, generalized anxiety disorders, and impulse control disorders are characterized by obsessive and/or compulsive behaviors. Obsessive behaviors typically include recurrent and persistent thoughts, impulses or images that occur over and over again and feel out of an individual's control. Compulsive behaviors include acts or compulsions that an individual performs over and over again, often according to certain rules. Obsessive compulsive disorders include general anxiety disorder, pathological or compulsive gambling disorders, compulsive eating, body dysmorphic disorders, hypochondriasis, pathological grooming conditions, kleptomania, pyromania, attention deficit hyperactivity disorder, and other impulse control disorders.

### EXAMPLES

The following examples are provided to illustrate embodiments of the present invention but are by no means intended to limit its scope.

### Example 1

### Administration of L-Methionine to Patients Experiencing Hot Flashes as Symptoms of Postmenopausal Hormone Variation

Patient No. 1

A 55 year old post-menopausal woman had experienced about 10 hot flashes per day for the past four years. After receiving gabapentin therapy for several days, she was unable to tolerate the drug due to severe dizziness.

L-methionine 1 gram tid was administered orally. After three weeks, a 90 percent improvement in hot flash frequency resulted. This benefit persisted for the 3 months of therapy. No side effects were experienced from L-methionine administration.

This patient had previously tried L-methionine at 500 mg three times daily for two weeks without noticing an appreciable change in her symptoms.

Patent No. 2

A 57 year old post-menopausal woman had experienced about 15 hot flashes per day for the past two years. L-methionine 1 g tid was administered orally and after three weeks of administration a 90 percent improvement in hot flash frequency resulted. This benefit persisted for the 5 months of therapy. No side effects were experienced from L-methionine administration.

Patent No. 3

A 57 year old post-menopausal woman had experienced about 7 hot flashes per day for the past two years. L-methionine 500 mg tid was administered orally. After three weeks of administration, a 66 percent improvement in nighttime hot flash frequency resulted and a 50 percent improvement in both nighttime and daytime hot flash severity resulted. No side effects were experienced from L-methionine administration.

Despite the absence of side effects, all three patients were instructed to discontinue L-methionine when a literature review revealed that high-dose oral L-methionine can increase serum homocysteine levels by 10-fold (van der Griend et al., Vasc. Med. 7:29-33 (2002), which is hereby incorporated by reference in its entirety). Since elevated serum homocysteine is associated with increased risks for development of cardiovascular disease, patients were told to discontinue L-methionine therapy.

Even though a potential side effect exists for L-methionine administration, the above examples demonstrate that L-methionine administration can be used to treat hot flashes and other symptoms of gonadal hormone variation resulting from menopause.

### Example 2

### Administration of L-Methionine to a Patient Experiencing Hot Flashes as a Result of the Post-Partum State

A 36 year old woman had experienced 3 severe nighttime and 2 moderate daytime hot flashes per day since giving birth to her first child 5 years ago. Nighttime hot flashes have been accompanied with severe sweating, requiring the patient to change her nighttime clothes three times a night. L-methionine 1 g bid was administered orally and after three weeks of administration, a 100 percent improvement in hot flash frequency resulted. No side effects were experienced from L-methionine administration.

Despite the absence of side effects, this patient was instructed to discontinue L-methionine when a literature review revealed that high-dose oral L-methionine can increase serum homocysteine levels by 10-fold (van der Griend et al., Vasc. Med. 7:29-33 (2002), which is hereby incorporated by reference in its entirety). Since elevated serum homocysteine is associated with increased risks for development of cardiovascular disease, this patient was told to discontinue L-methionine therapy.

Even though a potential side effect exists for L-methionine administration, the above example demonstrates that L-methionine administration can be used to treat hot flashes and other symptoms of gonadal hormone variation resulting from postpartum state.

### Example 3

### Administration of L-Methionine to a Patient Experiencing Palmar Hyperhidrosis

A 35 year old man with palmar hyperhidrosis for 20 years experienced a 50% reduction in the subjective severity of his condition after 3 weeks of therapy with L-methionine 1 g bid administered orally. No side effects were experienced from L-methionine administration.

Despite the absence of side effects, this patient was instructed to discontinue L-methionine when a literature review revealed that high-dose oral L-methionine can increase serum homocysteine levels by 10-fold (van der Griend et al., Vasc. Med. 7:29-33 (2002), which is hereby incorporated by reference in its entirety). Since elevated serum homocysteine is associated with increased risks for development of cardiovascular disease, this patient was told to discontinue L-methionine therapy.

Even though a potential side effect exists for L-methionine administration, the above example demonstrates that L-methionine administration can be used to treat palmar hyperhidrosis.

### Example 4

### Administration of L-Norleucine to Patients Experiencing Hot Flashes as Symptoms of Postmenopausal Hormone Variation

Patient No. 1

A 57 year old female with 6 hot flashes/day at baseline experienced a 70% reduction in hot flashes starting 10 days after initiating oral therapy with L-norleucine 1.5 grams, 2×/day. This level of efficacy was maintained for the 3.5 months of therapy. No side effects were experienced.

Patient No. 2

A 61 year old female with 8 hot flashes/day at baseline experienced an 87% reduction in hot flashes starting 4 days after initiating oral therapy with L-norleucine 1 gram, 2×/day. This level of efficacy was maintained for the 1.5 months of therapy. No side effects were experienced.

The above examples demonstrate that L-norleucine administration can be used to treat hot flashes and other symptoms of gonadal hormone variation resulting from menopause.

Although the invention has been described in detail for the purposes of illustration, it is understood that such detail is solely for that purpose, and variations can be made therein by those skilled in the art without departing from the spirit and scope of the invention which is defined by the following claims.

1. A method of treating a patient for a condition characterized by symptoms that can be alleviated by interfering with the activity of endogenous ligands on the α₂δ subunit of a voltage gated calcium channel, said method comprising:
administering to a patient experiencing the condition an amount of one or more of L-norleucine, L-isoleucine, L-alloisoleucine, L-methionine, L-leucine, 2-cyclohexylglycine, 2-phenylglycine, 2-amino-2-norbornane carboxylic acid, 1-aminocyclohexane carboxylic acid, 2-aminoheptanoic acid, 2-aminocaprylic acid, and 2-aminononanoic acid under conditions effective to treat the condition,
wherein when the condition is a hot flash or a symptom of hormonal variation, the compound is not L-leucine.

2. The method according to claim 1 wherein the compound is L-norleucine.

3. The method according to claim 1 wherein the compound is L-isoleucine.

4. The method according to claim 1 wherein the compound is L-alloisoleucine.

5. The method according to claim 1 wherein the compound is L-methionine.

6. The method according to claim 1 wherein the compound is L-leucine.

7. The method according to claim 1 wherein the compound is 2-cyclohexylglycine.

8. The method according to claim 1 wherein the compound is 2-phenylglycine.

9. The method according to claim 1 wherein the compound is 2-amino-2-norbornane carboxylic acid.

10. The method according to claim 1 wherein the compound is 1-aminocyclohexane carboxylic acid.

11. The method according to claim 1 wherein the compound is 2-aminoheptanoic acid.

12. The method according to claim 1 wherein the compound is 2-aminocaprylic acid.

13. The method according to claim 1 wherein the compound is 2-aminononanoic acid.

14. The method according to claim 1 wherein compound is administered in an amount of about 10 to about 5000 mg per day.

15. The method according to claim 1 wherein said administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, or by application to mucous membranes.

16. The method according to claim 1 wherein the compound is present in a pharmaceutical composition comprising the compound and a pharmaceutically-acceptable carrier.

17. The method according to claim 16 wherein the pharmaceutical composition is in a liquid or solid dosage form.

18. The method according to claim 1 wherein the compound is present in a nutritional supplement comprising the compound and an organoleptically suitable carrier.

19. The method according to claim 18 wherein the nutritional supplement is in a liquid or solid dosage form.

20. The method according to claim 1 wherein the condition is one or more of hot flashes or symptoms of hormonal variation, seizures, vertigo, migraine headaches, chronic pain disorders, a neurodegenerative disease, tic disorders, tremor disorders, nausea, cough, hiccups, asthma, hyperhidrosis, sleep disorders, fatigue, fibromyalgia, premature labor, preeclampsia or eclampsia, irritable bowel syndrome, inflammatory bowel disease, gastrointestinal damage caused by drugs and alcohol, drug addiction, obsessive compulsive disorders, generalized anxiety disorders, impulse control disorders, and attention deficit hyperactivity disorder.

21-46. (canceled)

47. A composition in a single unit dosage form comprising:
a pharmaceutically or organoleptically acceptable carrier and
one or more compounds selected from the group consisting of 2-cyclohexylglycine, 2-phenylglycine, 2-amino-2-norbornane carboxylic acid, 1-aminocyclohexane carboxylic acid, 2-aminoheptanoic acid, 2-aminocaprylic acid, 2-aminononanoic acid, L-norleucine, L-isoleucine, L-alloisoleucine, L-methionine, and L-leucine,
wherein the single unit dosage form comprises an amount of the one or more compounds which is effective to treat a condition characterized by symptoms that can be alleviated by interfering with the activity of endogenous ligands on the α₂δ subunit of a voltage gated calcium channel.

48. The composition according to claim 47 wherein the composition comprises two or more compounds.
